(19) United States
(12) Patent Application Publication
(10) Pub. No.: US 2025/0224409 A1
(43) Pub. Date: Jul. 10, 2025

(54) MINIATURIZED HANDHELD ELECTROCHEMICAL-BIOSENSOR SYSTEM FOR DETECTING CREATININE FROM SERUM SAMPLES AND ITS METHOD THEREOF

(71) Applicants: BIRLA INSTITUTE OF TECHNOLOGY AND SCIENCE, PILANI, Pilani (IN); BITS BioCyTiH Foundation, Jhunjhunu (IN)

(72) Inventors: Sanjeet Kumar, Hyderabad (IN); Satish Kumar Dubey, Hyderabad (IN); Sanket Goel, Hyderabad (IN)

(21) Appl. No.: 19/095,420

(22) Filed: Mar. 31, 2025

(30) Foreign Application Priority Data
Jun. 21, 2024 (IN) 202411047967

(51) Int. Cl. G01N 33/70; G01N 27/30; G01N 27/327; G01N 35/00

(52) U.S. Cl. CPC G01N 33/70; G01N 27/301; G01N 27/3278; G01N 35/00722; G01N 2035/0091

(57) ABSTRACT

The miniaturized handheld electrochemical biosensor system for detecting creatinine from serum samples. The system comprises a laser-induced graphene (LIG) device to collect and retain serum samples, said LIG device comprises: a flexible PI Sheet substrate; and at least three electrodes containing a reference electrode, a counter electrode, and a working electrode fabricated using a one-step fabrication process involving CO₂ Laser ablation on the polyimide sheet with optimized speed and power parameters; a portable potentiostat having a conducting tract to electrically connect the LIG device to the portable potentiostat to detect creatinine in serum samples within a range of 60 μM/L to 110 μM/L for males and 45 to 90 μM/L for females in serum samples using an indirect electrochemical method; and a graphical user interface coupled to a user computing device connected to the portable potentiostat to generate and display a graph upon receiving detected creatinine values from the portable potentiostat.

### CROSS-REFERENCE TO RELATED APPLICATION

This application claims the benefit of and priority to Indian patent application Ser. No. 20/241,1047967 filed Jun. 21, 2024, the contents of which being incorporated by reference in their entirety herein.

### TECHNICAL FIELD

The present disclosure relates to biosensor technology, specifically focusing on miniaturized handheld electrochemical biosensor systems designed for the detection of creatinine from serum samples. The disclosure encompasses both the hardware components and the methodology involved in utilizing such systems for efficient and accurate creatinine detection.

### BACKGROUND

Chronic kidney disease (CKD) is a condition in which the kidneys have become damaged over time and have a hard time doing all their important jobs, such as removing natural waste products and extra water from the body, helping make red blood cells, balancing important minerals in the body, helping maintain blood pressure, and keeping bones healthy. One important indicator of kidney function is the concentration of creatinine in the blood, which is a waste product that comes from the digestion of protein in food and the normal breakdown of muscle tissue. The serum (blood) creatinine test is a blood test used to check how well the kidneys are filtering the blood. Elevated levels of creatinine in the blood can be a sign of possible kidney problems, and the test is often used to check kidney health in people at high risk for CKD or for people with symptoms of acute kidney disease. The electrochemical approach is a promising technology for detecting creatinine levels in biological samples, offering a rapid, sensitive, and cost-effective method for detecting creatinine levels in biological samples. CKD is a common condition 850 million people worldwide and is a potent risk factor for kidney failure, heart disease, and death. The cause of CKD varies, but some of the most common factors include diabetes, high blood pressure, and cardiovascular disease. Managing CKD is focused on managing the disease(s) or condition(s) that are most likely causing the CKD, taking steps to slow down the CKD disease process directly, lowering the risk of cardiovascular disease, and treating any complications that may arise. The current standard for urine creatinine quantification uses a benchtop chemistry analyzer, which is expensive and large. A number of different chemistries for the detection of creatinine can be used, including the Jaffe reaction described below. A creatinine range 0.5-1.0 mg/dL (about 45-90 μmol/L) for women and 0.7-1.2 mg/dL (60-110 μmol/L) for men is normal. The creatinine is tightly correlated with the 24-hour urine result and is highly predictive of outcomes in multiple clinical settings, including, but not limited to, kidney disease, heart failure, cancer, and diabetes. Routine diagnosis and staging of chronic kidney disease (CKD), therefore, requires an evaluation of the creatinine in the patient's serum and blood chemistry analysis to estimate GFR.

The current standard for serum creatinine quantification uses a benchtop chemistry analyzer, which is expensive and large. Several chemistries for detecting creatinine can be used, including the Jaffe reaction described below. While inexpensive point-of-care methods (e.g., dipstick tests) do exist and are widely available for detecting serum creatinine, the limit of detection (LOD) and accuracy of these dipstick tests is usually not good. Therefore, they are not sufficiently quantitative and lack adequate precision. Inexpensive point of care tests for serum creatinine do not exist. A POC device and method for detecting and measuring creatinine levels in a serum sample of a subject that optimally balances accuracy, cost, simplicity and low facility requirement would be a highly desirable tool to effectively address the epidemic of CKD worldwide.

Traditional methods for quantifying serum creatinine often involve costly and bulky benchtop chemistry analyzers, posing limitations in terms of accessibility and efficiency. While inexpensive point-of-care tests exist, they often lack sufficient accuracy and precision, hindering their effectiveness in clinical settings. An electrochemical approach emerges as a promising technology for detecting creatinine levels in biological samples, offering rapid, sensitive, and cost-effective results. However, existing methodologies still face challenges in achieving optimal balance between accuracy, cost-effectiveness, simplicity, and facility requirements. Addressing these limitations, a need arises for innovative methodologies and devices that enable accurate and efficient detection and measurement of creatinine levels in serum samples. Such advancements would not only enhance diagnostic capabilities but also facilitate early intervention and management of CKD, thereby mitigating its adverse health outcomes.

Furthermore, attempts have been made to develop a portable device, the LIG electrode platform, to detect serum creatinine samples, but many of them are limited in selectivity, interference issues, complex sample pre-treatment, and portability challenges. Conventional approaches to serum creatinine detection typically involve taking large sample volumes (in milliliters) from the patient, which can be inconvenient and uncomfortable for the patient. This approach may also lead to increased risk of contamination and sample degradation. The conventional diagnostics process for serum creatinine detection consumes a significant amount of time, from sample collection to obtaining the diagnostic report. Many existing devices for serum creatinine detection lack point-of-care testing capabilities. The devices are not truly portable as most of the devices required (reagents and enzyme immobilization) have more power requirements and cannot function autonomously. Lack of True Portability: Existing devices for serum creatinine detection often lack true portability due to their reliance on external power sources and the need for additional reagents and enzyme immobilization.

In view of the foregoing discussion, it is portrayed that there is a need to have a system and method that allow for the detection and measurement of creatinine in a serum sample of a subject and address the disadvantages of existing technologies.

### BRIEF SUMMARY

The present disclosure seeks to provide an innovative, automated, and portable detection system for creatinine utilizing electrochemical devices. The primary objectives include the creation of miniaturized electrodes and implementing a one-step fabrication process specifically tailored for Graphene Electrodes. The aim is to revolutionize diagnostics by introducing a Point of Care application, enabling rapid and efficient creatinine detection directly at the patient's side. The proposed system emphasizes miniaturization, using miniaturized electrodes for enhanced efficiency and portability. Furthermore, it integrates a sample-to-answer approach, streamlining the diagnostic process for seamless and user-friendly operation. The deployment of a handheld device ensures end-user accessibility, facilitating ease of use in various clinical settings. Additionally, incorporating machine learning (ML) techniques plays a crucial role in enhancing sensor data quality and accuracy. ML integration allows for real-time data analysis and interpretation, ultimately improving diagnostic outcomes. Overall, this innovative approach is promising in advancing point-of-care creatinine detection, offering a comprehensive solution for efficient and reliable diagnostics.

In an embodiment, a miniaturized handheld electrochemical biosensor system for detecting creatinine from serum samples is disclosed. The system includes a LIG (laser-induced graphene) device to collect and retain serum samples, the LIG device comprises a flexible PI Sheet substrate, and at least three electrodes containing a reference electrode, a counter electrode, and a working electrode fabricated using a one-step fabrication process involving CO2 Laser ablation on the polyimide sheet with optimized speed and power parameters.

The system further includes a portable potentiostat having a conducting tract to electrically connect the LIG device to the portable potentiostat to detect creatinine in serum samples within a range of 60 μM/L to 110 μM/L for males and 45 to 90 μM/L for females in serum samples using an indirect electrochemical method.

The system further includes a graphical user interface coupled to a user computing device connected to the portable potentiostat to generate and display a graph upon receiving detected creatinine values from the portable potentiostat.

In another embodiment, a method for detecting creatinine from serum samples is disclosed. The method includes providing a sample containing creatinine.

The method further includes applying 10 μL sample droplet onto a surface of LIG electrodes of a LIG (laser-induced graphene) device.

The method further includes establishing an electrical connection between the LIG electrodes and a portable detection system.

The method further includes detecting creatinine in serum samples received from the LIG device through a conducting tract within a range of 60 μM/L to 110 μM/L for males and 45 to 90 μM/L for females in serum samples using an indirect electrochemical method using a portable potentiostat.

The method further includes generating a graph using a graphical user interface upon receiving detected creatinine values from the portable potentiostat thereby displaying on a user computing device.

An object of the present disclosure is to develop a portable device, the LIG electrode platform, to detect serum creatinine samples, with this device increased selectivity sensitivity also simplified sample preparation process.

Another object of the present disclosure is to revolutionize kidney disease diagnosis and management, ultimately improving patient outcomes and reducing healthcare burdens worldwide.

Another object of the present disclosure is to develop simple, low-cost, robust, portable, miniaturized electrodes alternate for detection abilities and ML integration to increase the sensor performance.

Yet another object of the present disclosure is to deliver an expeditious and cost-effective device operating on a commercially available portable potentiostat attached to a mobile and LIG-based test strip electrode to detect creatinine using a microliter of serum sample.

To further clarify the advantages and features of the present disclosure, a more particular description of the disclosure will be rendered by reference to specific embodiments thereof, which are illustrated in the appended drawings. It is appreciated that these drawings depict only typical embodiments of the disclosure and are therefore not to be considered limiting of its scope. The disclosure will be described and explained with additional specificity and detail in the accompanying drawings.

### BRIEF DESCRIPTION OF FIGURES

These and other features, aspects, and advantages of the present disclosure will become better understood when the following detailed description is read concerning the accompanying drawings in which like characters represent like parts throughout the drawings, wherein:

FIG. 1 illustrates a block diagram of a miniaturized handheld electrochemical biosensor system for detecting creatinine from serum samples, in accordance with an embodiment of the present disclosure;

FIG. 2 illustrates a flow chart of a method for detecting creatinine from serum samples, in accordance with an embodiment of the present disclosure;

FIG. 3(a) illustrates a schematic diagram that shows three-electrode arrangement, polyimide substrate (PI Substrate) in accordance with an embodiment of the present disclosure;

FIG. 3(b) illustrates the electrochemically active various modification on the electrode, in accordance with an embodiment of the present disclosure;

FIG. 4(a) illustrates a schematic exploded illustrate of three electrode systems of the electrochemically active indirect creatinine detection in accordance with an embodiment of the present disclosure;

FIG. 4(b) illustrates a various modification system, in accordance with an embodiment of the present disclosure;

FIG. 5 illustrates an exemplary profile of the LIG Electrode with the overall dimension on the PI substrate, in accordance with an embodiment of the present disclosure;

FIG. 6 illustrates a single-step fabrication process for three three-electrode electrochemically systems, in accordance with an embodiment of the present disclosure;

FIG. 7 illustrates a schematic diagram for the smartphone assembly, a portable electrochemical analyzer, and three electrode detection system, in accordance with an embodiment of the present disclosure;

FIG. 8 illustrates a final device for detecting the creatinine is (LIG Electrode system, a sensit smart portable system and smartphone for data interpretation), in accordance with an embodiment of the present disclosure;

FIG. 9(a) illustrates the responses for the creatinine concentration range (0 to 200 μM) at the scan rate of the 50 mV/s;

FIG. 9(b) illustrates the calibration plot for the same having the (R²=0.89), in accordance with an embodiment of the present disclosure;

FIG. 10(a) illustrates the creatinine concentration perdition using the linear regression model, in accordance with an embodiment of the present disclosure;

FIG. 10(b) illustrates ML original data set versus predicted model having the R² Score value is 0.99, in accordance with an embodiment of the present disclosure;

FIG. 11 illustrates a real image of the device with the overall dimensions, in accordance with an embodiment of the present disclosure;

FIG. 12 illustrates the various techniques for the creatinine concentration prediction and R² Score value, in accordance with an embodiment of the present disclosure;

FIG. 13 illustrates a block diagram of the Miniaturized Handheld Electrochemical Analyzer for the detection of creatinine, in accordance with an embodiment of the present disclosure;

FIG. 14 illustrates a Table depicting components and their functions, in accordance with an embodiment of the present disclosure; and

FIG. 15 illustrates a flow chart of the disclosure, in accordance with an embodiment of the present disclosure.

Further, skilled artisans will appreciate those elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

To promote an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail concerning the accompanying drawings.

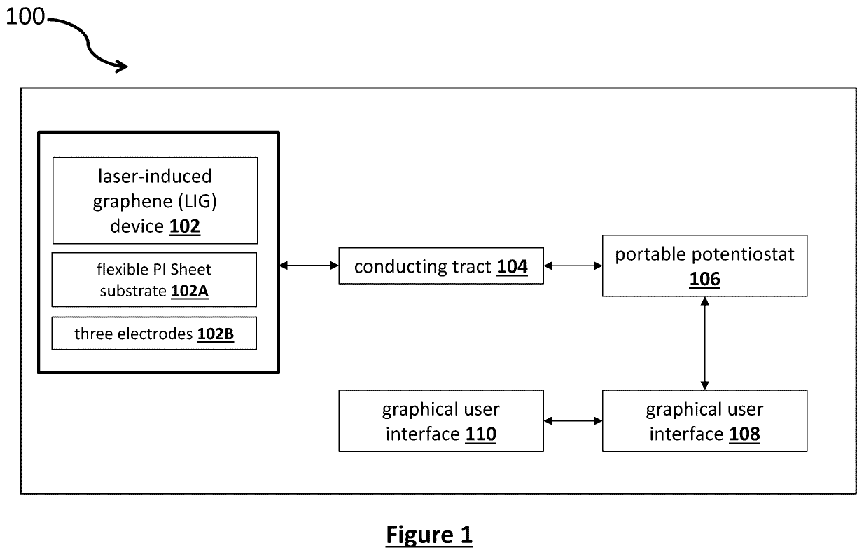

Referring to FIG. 1, a block diagram of a miniaturized handheld electrochemical biosensor system for detecting creatinine from serum samples is illustrated in accordance with an embodiment of the present disclosure. The system 100 includes a LIG (laser-induced graphene) device (102) to collect and retain serum samples. The LIG device (102) comprises a flexible PI Sheet substrate (102A), and at least three electrodes (102B) containing a reference electrode, a counter electrode, and a working electrode fabricated using a one-step fabrication process involving CO₂ Laser ablation on the polyimide sheet with optimized speed and power parameters.

In an embodiment, a portable potentiostat (106) is having a conducting tract (104) to electrically connect the LIG device (102) to the portable potentiostat (106) to detect creatinine in serum samples within a range of 60 μM/L to 110 μM/L for males and 45 to 90 μM/L for females in serum samples using an indirect electrochemical method.

In an embodiment, a graphical user interface (110) is coupled to a user computing device (108) connected to the portable potentiostat (106) to generate and display a graph upon receiving detected creatinine values from the portable potentiostat (106).

In one embodiment, the reference electrode is modified with silver-silver chloride ink (Ag/AgCl), the counter electrode and working electrode are modified with gold nanoparticles (AuNPs), and a picrate solution is placed after the modification of the working electrodes.

In another embodiment, the laser-induced graphene electrodes are obtained using a one-step fabrication process, wherein each graphene electrode comprises a substrate holder configured to hold a substrate. In one embodiment, a laser source is configured to emit a laser beam. In one embodiment, a beam controller is configured to control the laser beam and direct it onto the substrate. In one embodiment, a processing unit is configured to control the laser source and beam controller to perform laser scribing on the substrate for converting a portion of the substrate into graphene.

In a further embodiment, the substrate comprises a polymer material, wherein the polymer material comprises polyimide.

The system further comprises a gas delivery unit configured to provide an inert atmosphere around the laser scribing region. In one embodiment, a temperature control unit is configured to maintain a desired temperature during the laser scribing process.

Yet, in another embodiment, a machine learning technique is incorporated to increase sensing and performance of the electrode with the various techniques selected from a group of regression-based accuracy matrices such as decision tree (DT), Linear Regression (LR), random forest (RF), K-Nearest Neighbor (kNN) and AdaBoost model.

Yet, in one embodiment, the LIG device (102) contains enzymeless indirect sensing chemicals containing Picric acid and Sodium Hydroxide NaOH Solution.

In another embodiment, the enzyme-less detection method works in a picrate anion consumed upon reaction with creatinine.

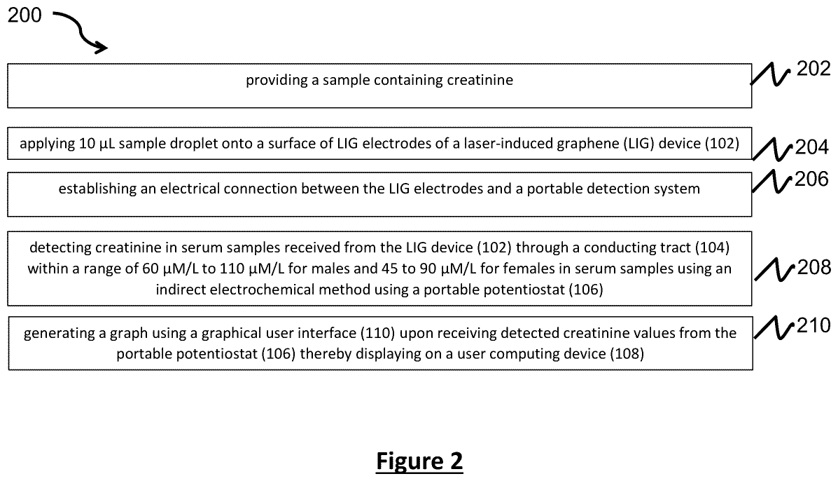

FIG. 2 illustrates a flow chart of a method for detecting creatinine from serum samples, in accordance with an embodiment of the present disclosure. At step 202, method 200 includes providing a sample containing creatinine.

At step 204, method 200 includes applying 10 μL sample droplet onto a surface of LIG electrodes of a LIG (laser-induced graphene) device (102).

At step 206, method 200 includes establishing an electrical connection between the LIG electrodes and a portable detection system.

At step 208, method 200 includes detecting creatinine in serum samples received from the LIG device (102) through a conducting tract (104) within a range of 60 μM/L to 110 μM/L for males and 45 to 90 μM/L for females in serum samples using an indirect electrochemical method using a portable potentiostat (106).

At step 210, method 200 includes generating a graph using a graphical user interface (110) upon receiving detected creatinine values from the portable potentiostat (106) thereby displaying on a user computing device (108).

The method further comprises fabricating miniaturized electrodes for creatinine detection, comprising the steps of providing a substrate, such as a flexible polyimide sheet. Then, directing a laser beam onto the substrate to perform laser scribing. Then, controlling the laser scribing process to convert a portion of the substrate into graphene, wherein the laser scribing process is performed in an inert atmosphere. Thereafter, patterning the graphene on the substrate by controlling the movement of the laser beam.

In a further embodiment, the laser scribing process comprises controlling the laser power to achieve a desired level of conversion of the substrate to graphene. Then, controlling the scan speed of the laser beam to achieve a desired quality of the graphene. Then, controlling the ambient temperature during the laser scribing process. Then, optimizing the laser scribing process parameters to control the number of graphene layers formed. Thereafter, optimizing the laser scribing process parameters to minimize defects and ensure uniformity in the graphene layer.

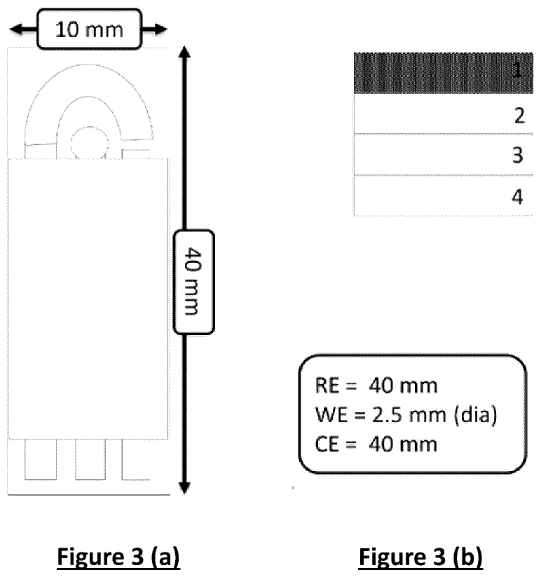

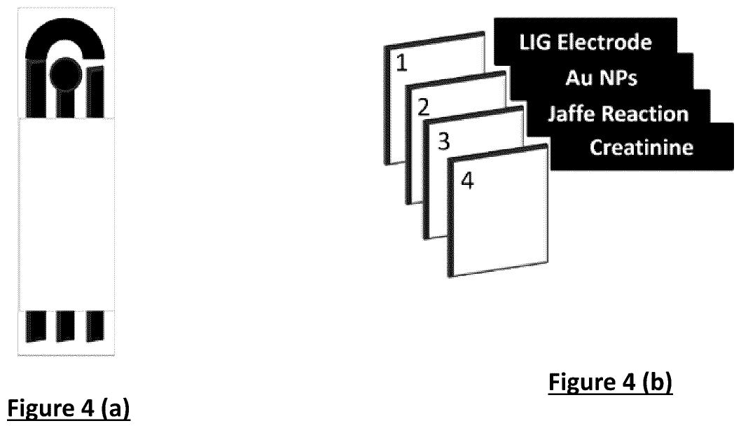

FIG. 3 illustrates (a) a schematic diagram that shows three-electrode arrangement, polyimide substrate (PI Substrate), (b) the electrochemically active various modification on the electrode, in accordance with an embodiment of the present disclosure.

Accordingly, the present disclosure provides an electrochemical biosensor to detect creatinine in biological samples. For the present disclosure, the generally accepted creatinine in the range of the 60 μM/L to 110 μM/L for males and 45 to 90 μM/L for females in serum samples.

An electrochemically active Laser-Induced electrode device for collecting and retaining a serum sample is provided with conducting that are ablated on the polyimide substrate. The electrodes consist of the three electrodes system. The working electrodes are modified with the gold nanoparticle for good electrocatalytic properties enhancement.

An electrode holder for holding the electrochemical device with a portable detection system (sensit-smart portable Potentiostat).

A simple smartphone-based detection system using electrochemically laser-induced based active electrodes systems.

An indirect electrochemical method or enzymeless detection method has been developed. An enzyme-less detection method works ion the picrate anion consumed upon the reaction with creatinine and the gold-modified Laser-induced graphene-based electrode has been deployed for the first time.

A point of care biosensor for the detection of the creatinine concentration of the biological sample is provided.

A microliter sample volume is provided for the serum sample.

FIG. 4 illustrates (a) a schematic exploded illustrate of three electrode systems of the electrochemically active indirect creatinine detection (b) a various modification system, in accordance with an embodiment of the present disclosure.

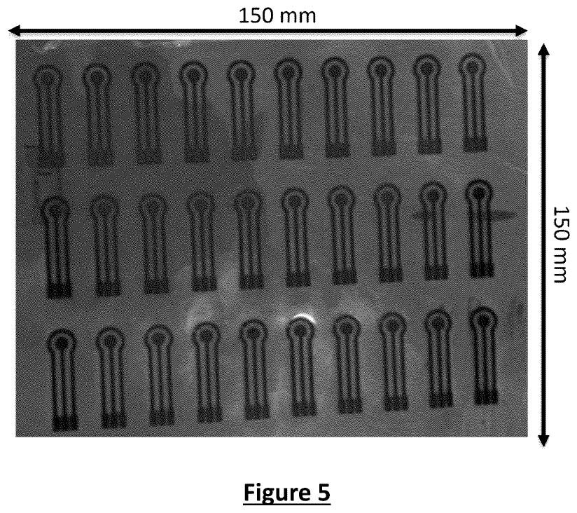

FIG. 5 illustrates an exemplary profile of the LIG Electrode with the overall dimension on the PI substrate, in accordance with an embodiment of the present disclosure.

### Fabrication Flow Diagram:

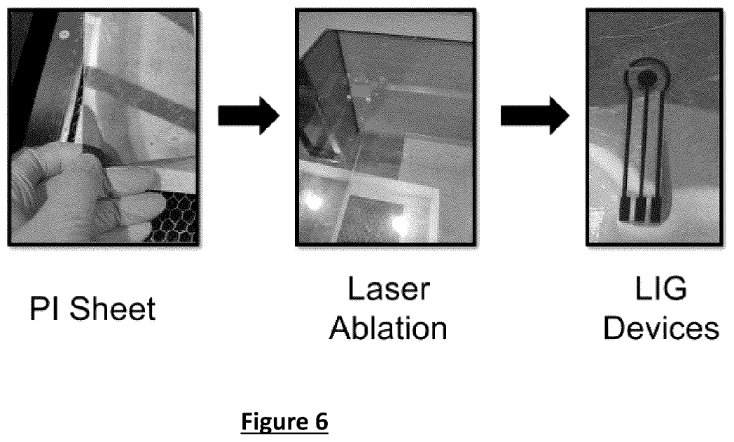

A single-step electrode fabrication method. The substrate material is made up of polymer composition, PI Sheet having a flexible nature. (Inset real image of the LIG test strip shown in FIG. 5). A CO₂ Laser ablation method is introduced on the polyimide sheet with optimized speed and power. For the fabrication of the LIG electrochemically device, as shown in FIG. 6.

FIG. 6 illustrates a single-step fabrication process for three three-electrode electrochemically systems, in accordance with an embodiment of the present disclosure.

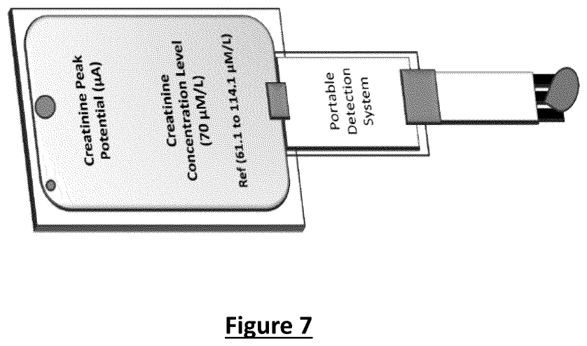

FIG. 7 illustrates a schematic diagram for the smartphone assembly, a portable electrochemical analyzer, and three electrode detection system, in accordance with an embodiment of the present disclosure.

### Portable Creatinine Detection System

A three-electrode system shown in FIG. 7 is electrically connected to the conducting tract (104) to the portable detection system. The electrodes consist of the counter, working, and reference. The different conducting layers have various modifications. The electrochemically active substance used for detecting the creatinine in the serum sample is picrate anion consumed upon the reaction of the creatinine electrochemically measured.

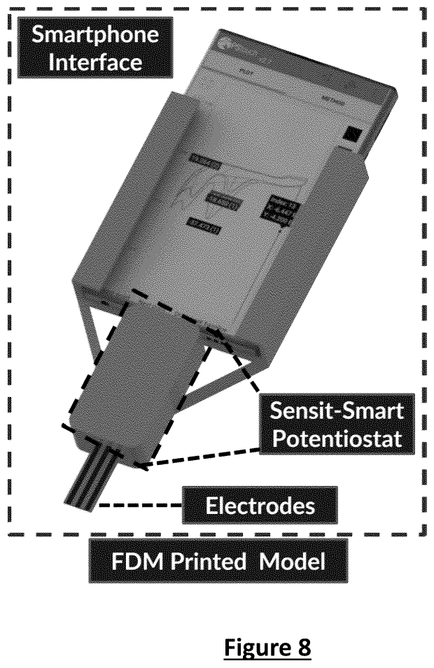

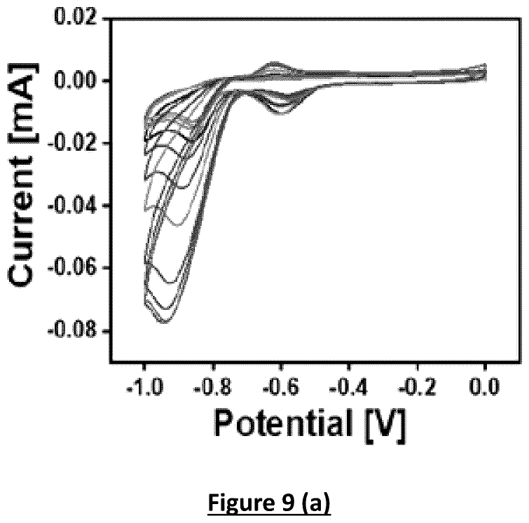

FIG. 8 illustrates a final device for detecting the creatinine is (LIG Electrode system, a sensit smart portable system and smartphone for data interpretation), in accordance with an embodiment of the present disclosure. The above final development of the portable device consists of a smartphone, Graphene-based Electrodes, commercially available sensit smart portable Potentiostat, electrode connector, enzymeless indirect sensing chemicals ([Picric acid and Sodium Hydroxide NaOH Solution pH 13), Gold nanoparticles (AuNPs). Herein, a portable potentiostat has been used to detect serum creatinine. The CHI 1030c has been used to validate the laser-induced graphene electrode using electrochemical methods (cyclic voltammetry techniques).

The miniaturized electrode system has been used to detect the creatinine level, smart phone-based application has been utilized to run the electrochemical techniques, and the cv technique has been used in the range of the (0 to −1 V) at the scan rate of 50 mV/s. The reduction peak potential is observed at −0.8 V, the electrochemical response (CV), which confirms the picrate anionic formed due to the creatinine.

The overall reaction time of miniaturized electrodes facilitates faster reaction kinetics, allowing for rapid electrochemical detection of creatinine, which takes two minutes to get the results.

Developing a miniaturized electrode and a portable detection system represents a significant advancement in the field. The compact design allows for easy transportation and deployment in various settings, including clinics, hospitals, and even remote locations where access to laboratory facilities may be limited.

Efficacy: Implementing a one-step fabrication process for Graphene Electrodes further enhances efficacy by simplifying manufacturing and ensuring consistent electrode quality, leading to more reliable performance in diagnostic applications.

Time: The proposed system aims to significantly reduce the time required for creatinine detection by employing a rapid, one-step process. Traditional methods often involve multiple steps and longer assay times, leading to delays in obtaining results.

Reactions: Integrating miniaturized electrodes facilitates faster reaction kinetics, allowing for rapid electrochemical detection of creatinine. This leads to shorter assay times and improved overall efficiency.

Further, the ML techniques are incorporated to increase the sensor efficiency and increase the performance of the electrode with the various techniques such as regression based accuracy matrices such as decision tree (DT), Linear Regression (LR), random forest (RF), K-Nearest Neighbor (kNN) and AdaBoost model.

Certainly! Here's an elaboration on the novelty of the proposed system in terms of portability, efficacy, time, and reactions:

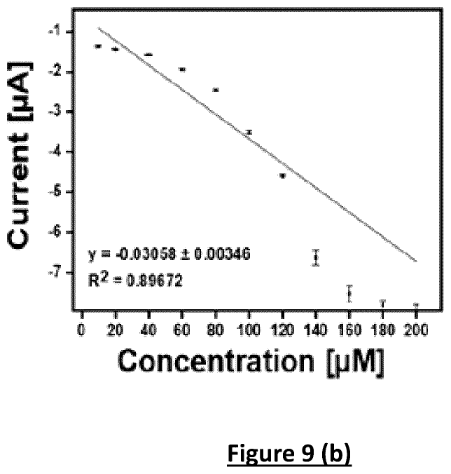

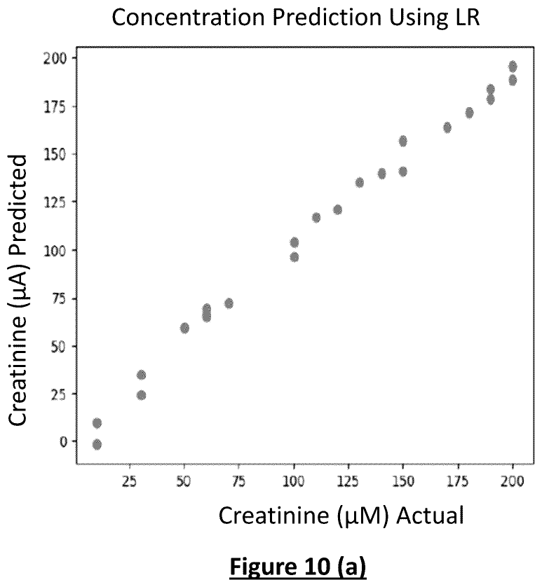

FIG. 9 illustrates (a) the responses for the creatinine concentration range (0 to 200 μM) at the scan rate of the 50 mV/s. (b) the calibration plot for the same having the (R²=0.89), in accordance with an embodiment of the present disclosure.

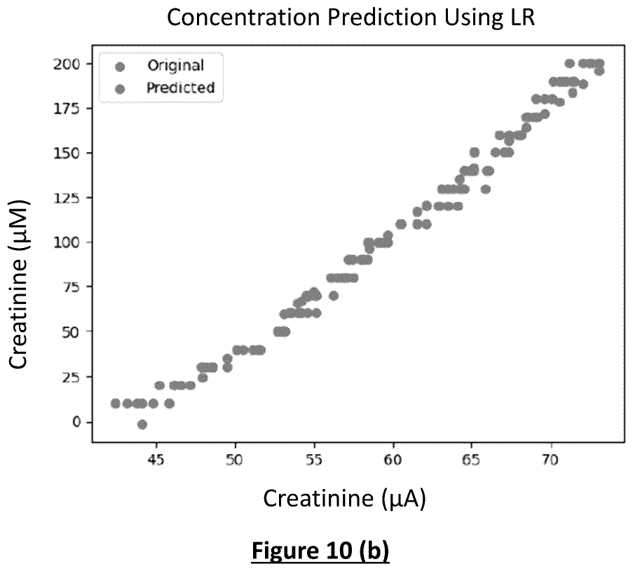

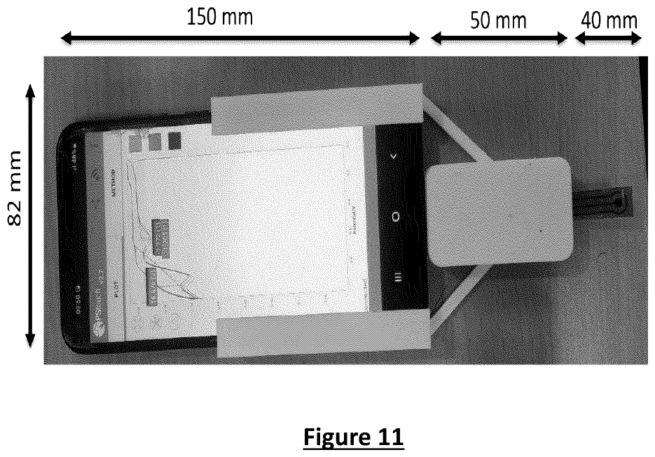

FIG. 10 illustrates (a) the creatinine concentration perdition using the linear regression model, (b) ML original data set versus predicted model having the R² Score value is 0.99, in accordance with an embodiment of the present disclosure.

The device can help detect a creatinine using low sample volume (5 microliters) with the smartphone-based electrochemical detection device. The device can help in rapid and real-time monitoring with the disposable electrode system. Smartphone-based miniaturized, portable device and replaceable electrode system for the point of source detection system without any battery uses, suitable for on-the-site deployment. The device can be developed with the Raspberry Pi platform for enhancing the processing and storing of data with integrated facilities. It can be correlated with system-on-a-chip (SoC) and lab-on-a-chip (LoC) platform executables for numerous point-of-source applications.

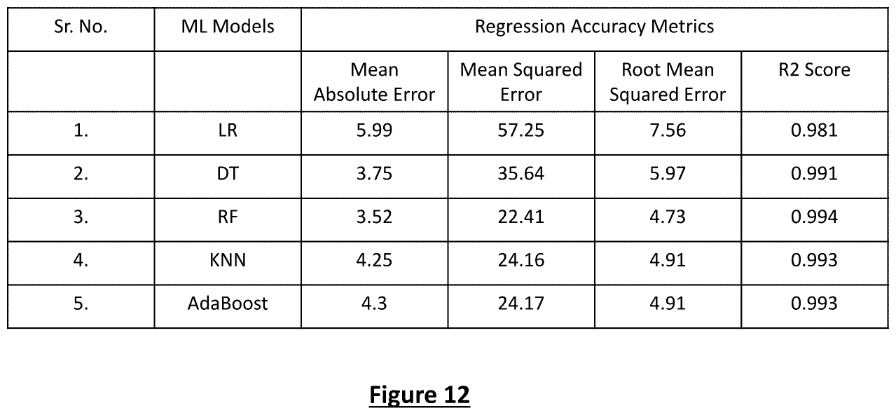

FIG. 11 illustrates a real image of the device with the overall dimensions, in accordance with an embodiment of the present disclosure.

In an embodiment, the detection of creatinine is performed with the help of electrochemical (Cyclic Voltammetry) techniques. The three-electrode system-based devices are fabricated using the polyimide sheet (PI). The reference electrodes are modified with silver-silver chloride ink (Ag/AgCl), the counter electrode and working electrode are modified with gold nanoparticles, and a picrate solution is also placed after the modification of the working electrodes. The laser-induced graphene electrodes are obtained using the single-step fabrication process. The CV response is measured in the range of (0 to −1 V) at the scan rate of 50 mV/s. The observed cv responses show the reduction peak potential at −0.8 V., which confirms that the picrate anionic formation is due to creatinine.

The ML techniques are incorporated to increase the sensor efficiency and increase the performance of the electrode with the various techniques such as regression-based accuracy matrices such as decision tree (DT), Linear Regression (LR), random forest (RF), K-Nearest Neighbor (kNN) and AdaBoost model.

FIG. 12 illustrates the various techniques for the creatinine concentration prediction and R² Score value, in accordance with an embodiment of the present disclosure.

A portable read out circuit for the voltammetry can be used to measure the sample. A machine learning model-based mobile application for the sensor accuracy measurement. A self-calibration feature can be introduced to the device. A non-invasive method can be deployed for accessible sample collection and to improve the repeatability and the detection limit (LOD).

The devices have been successfully tested with 15 different samples with the consent of the patients to detect creatinine with the serum sample using electrochemical. The data is stored in the form of a graph and analyzed.

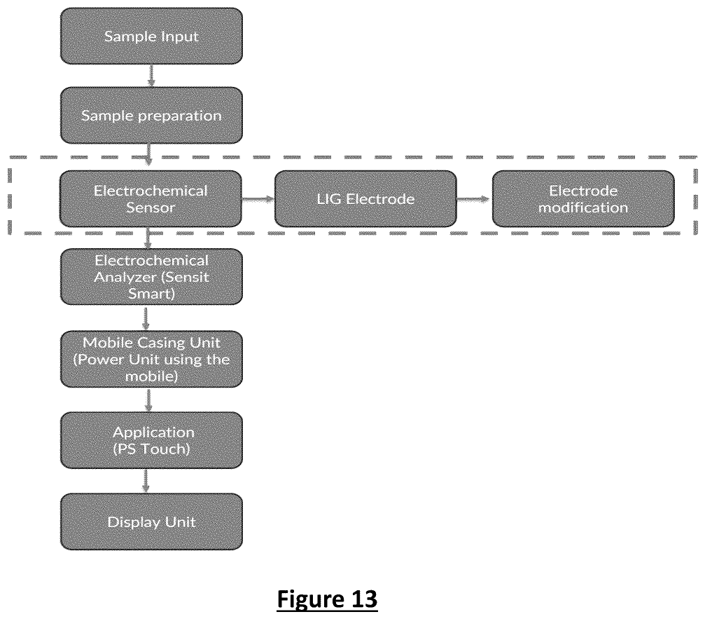

FIG. 13 illustrates a block diagram of the Miniaturized Handheld Electrochemical Analyzer for the detection of creatinine, in accordance with an embodiment of the present disclosure.

Laser-induced electrode devices represent a significant advancement in biomedical applications, particularly for serum sample collection and retention. These devices leverage precise laser processing to create efficient electrodes directly on a substrate, often a flexible polyimide sheet. The fabrication process begins with meticulous substrate preparation, including cleaning with isopropanol to eliminate organic contaminants. Subsequently, electrode designs are created using CAD tools like SolidWorks, specifying dimensions such as 40 mm×10 mm. The core of the fabrication involves laser ablation, employing optimized parameters (e.g., 6.5% power and 4.5% speed using a CO₂ laser) to form laser-induced graphene on the polyimide sheet, which serves as the conductive material due to its excellent electrical properties and biocompatibility.

The functioning of these laser-induced graphene electrodes in serum sample handling is multifaceted. For collection, a small serum drop (10 μL) is applied to the electrode's porous structure, which facilitates absorption. The graphene's electrochemical properties enable sensitive capture of serum biomolecules. Retention is achieved through the porous structure's large surface area, which physically holds the serum, and through adsorption, where biomolecules adhere to the graphene surface due to strong T-x interactions. Furthermore, functionalization with specific chemical groups, such as gold nanoparticles and picric acid, can enhance the selective retention of target analytes.

Finally, the collected and retained serum samples are analyzed using techniques like electrochemical analysis. By connecting the electrodes to an electrochemical analyzer, electrical signals generated from the interaction of serum components with the graphene surface are measured, enabling the detection of analytes like creatinine. This miniaturized electrode technology is crucial for high-precision applications in biomedical devices and sensors, offering a robust and efficient platform for serum sample management and analysis.

The one-step fabrication process for graphene electrodes simplifies the creation of these crucial components by integrating graphene synthesis and patterning into a single, streamlined procedure. This method typically employs laser scribing, where a carbon precursor, often a polymer like polyimide, is directly converted into graphene on a desired substrate. The process begins with the preparation of the polyimide substrate, selected for its flexibility, thermal stability, and compatibility with laser scribing. A focused laser beam, usually from a CO₂ laser, irradiates the substrate's surface, inducing a photothermal conversion that reorganizes the carbon atoms into graphene. Simultaneously, the laser's movement patterns the graphene into the required electrode shapes.

Key parameters are meticulously controlled to ensure optimal results. Laser parameters, including power (e.g., 6.5%), scan speed (e.g., 4.5%), and wavelength, are adjusted to balance complete conversion with substrate integrity. Substrate considerations, such as material and thickness, also play a vital role. Environmental conditions, like maintaining an inert atmosphere and controlling temperature, contribute to consistent processing. Furthermore, achieving high-quality graphene requires optimizing layer number and minimizing defects.

This one-step laser scribing method offers several advantages. Its simplicity and efficiency reduce fabrication time, while its scalability makes it suitable for large-scale production. The method's flexibility, particularly with substrates like polyimide, enables applications in wearable electronics and flexible devices. By precisely managing process parameters, high-quality graphene electrodes can be fabricated for various advanced applications.

Electrode performance can be significantly enhanced through modifications, notably the introduction of gold nanoparticles (AuNPs), which are employed to improve electrolytic properties. These modifications play a crucial role in optimizing the electrode's ability to facilitate electrochemical reactions, thereby improving its sensitivity and efficiency in various applications.

The practical application of these modified electrodes necessitates a well-designed electrode holder. Typically constructed from chemically inert and mechanically robust materials like polycarbonate or PTFE, or insulated metals such as stainless steel or aluminum, the holder ensures electrode stability. Its design incorporates a secure clamping or screw mechanism, precision-cut slots for consistent electrode positioning, and conductive electrical contacts (often gold-plated) for reliable signal transmission. Insulation is critical to prevent short-circuiting. Portability is also prioritized, with compact size and durability being key features. Functionally, the holder maintains electrode stability, ensures proper electrical connectivity, and simplifies electrode insertion and removal, facilitating efficient setup and teardown.

Complementing the electrode holder is the portable detection system, designed for on-site analysis in diverse environments. Its structure includes a robust, often waterproof and dustproof, enclosure to protect internal components. A user-friendly interface, typically a touchscreen or button-operated, streamlines operation. Battery-powered for portability, the system also allows for external power supply. Results are displayed in real-time, and data storage or transmission capabilities are included. This portable design enables on-site testing and analysis, making it ideal for field research, clinical diagnostics, and environmental monitoring, ensuring that the enhanced electrode performance can be effectively utilized in various practical settings.

The smartphone-based detection system seamlessly interfaces with electrochemically laser-induced active electrodes through a multi-step process, ensuring precise measurement and data collection. Initially, the active electrodes undergo preparation and functionalization using a laser-induced process, enhancing their electrochemical properties and creating a highly sensitive surface for analyte detection. The smartphone then integrates with a dedicated hardware module, typically including a potentiostat, which connects via USB or Bluetooth. When a sample is applied to the electrodes, an electrochemical reaction occurs, generating an electrical signal that is captured by the potentiostat. This signal is digitized and transmitted to the smartphone, where a specialized app, such as PS Touch, processes and displays the data, showing a graph of different creatinine concentrations. The app also enables real-time monitoring and data storage, with options for data sharing via cloud storage or direct transmission to healthcare providers.

Validation of these electrodes involves rigorous testing to assess performance metrics like sensitivity, specificity, stability, and reproducibility. Cyclic voltammetry (CV) is employed to evaluate the electrochemical activity and reversibility of the laser-induced graphene (LIG) electrodes. A potential sweep is applied, and the resulting current is measured, providing a cyclic voltammogram that reveals the redox behavior of the electrode surface. Peaks in the voltammogram are analyzed to determine the electrochemical properties. Real sample analysis validates the practical applicability of the electrodes by testing them with biological fluids and comparing the results with standard methods. Reproducibility and repeatability tests ensure consistent performance across multiple tests and batches, with statistical analysis used to determine the variability of the electrode performance.

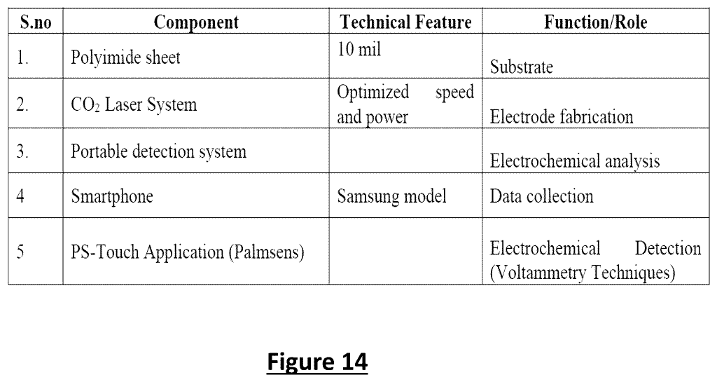

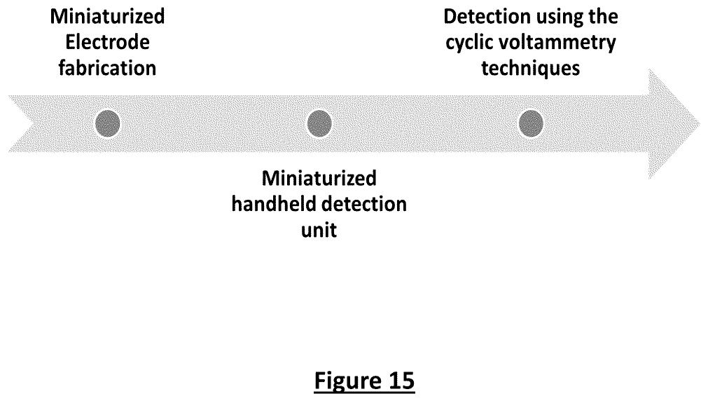

FIG. 14 illustrates a Table depicting components and their functions, in accordance with an embodiment of the present disclosure. FIG. 15 illustrates a flow chart of the disclosure, in accordance with an embodiment of the present disclosure.

The miniaturized electrode system provides substantial advantages over traditional methods, particularly in reaction kinetics and detection speed. These benefits arise from the unique properties and design of the miniaturized electrodes. The increased surface area-to-volume ratio enhances electron transfer rates, leading to faster reaction kinetics. Additionally, reduced diffusion distances expedite analyte-electrode interaction, accelerating the overall reaction process. The system also boasts rapid response times and high sensitivity, enabling quicker detection of analytes, even at low concentrations. The reduced capacitance of miniaturized electrodes minimizes background noise, improving the signal-to-noise ratio and ensuring clearer, faster detection.

Furthermore, the system promotes efficient reagent use, requiring lower sample volumes and minimizing reagent consumption, which reduces costs. The compact design facilitates on-site point-of-care testing, crucial for applications needing immediate results, such as clinical settings. Integration with portable devices like smartphones enables rapid data acquisition and real-time analysis. The system's scalability and flexibility make it suitable for high-throughput screening and customization for diverse detection needs.

The significant reduction in assay time achieved by this system offers numerous benefits compared to traditional assay methods. This reduction in time is critical for applications where rapid results are essential, such as in emergency medical situations or environmental monitoring. Faster assay times allow for quicker diagnoses and interventions, improving patient outcomes and decision-making processes. The efficiency gained from reduced assay times also increases throughput, enabling the analysis of more samples within a given timeframe. This is particularly valuable in research and industrial settings where large sample volumes need to be processed. Moreover, the reduced assay time often translates to lower operational costs, as it minimizes the need for extensive labor and resources.

The rapid results provided by the proposed system are essential for timely decision-making, particularly in medical diagnostics where faster diagnoses can significantly improve patient outcomes. In emergency situations, such as infectious disease outbreaks, quick assay times are critical for immediate response and containment. Furthermore, the reduced assay time increases throughput, enabling the analysis of more samples within a shorter period. This is beneficial in laboratories and large-scale screening programs, where high volumes of samples need to be processed efficiently.

The one-step fabrication process for graphene electrodes offers significant advantages in consistency and reliability. By simplifying manufacturing and eliminating multiple fabrication stages, the process reduces complexity and minimizes variability. This streamlined approach ensures uniform electrode properties across different batches, promoting consistent material quality and controlled fabrication conditions. High reproducibility is achieved through batch-to-batch consistency and reduced human error, resulting in stable electrochemical behavior, durability, and longevity. The simplicity and efficiency of the one-step process also facilitate scalability and cost-effectiveness, enabling easier scaling up of production without compromising quality.

Machine learning (ML) algorithms further enhance sensor efficiency and performance by optimizing data analysis, improving accuracy, and enabling real-time processing. ML algorithms efficiently process large volumes of sensor data, identifying patterns and extracting relevant features for accurate diagnostics. By distinguishing between signal and noise, ML algorithms improve the signal-to-noise ratio, leading to more reliable sensor readings. Advanced ML algorithms enable real-time data analysis, providing immediate diagnostic feedback and facilitating timely decision-making. These improvements contribute to more accurate and efficient diagnostic outcomes.

Machine learning (ML) algorithms significantly enhance sensor performance through various mechanisms. They can automatically calibrate sensors and correct for drifts or inconsistencies, ensuring consistent performance over time. Through sophisticated pattern recognition and anomaly detection, ML algorithms improve sensor sensitivity and specificity, enabling more accurate detection of target analytes. Specific ML algorithms such as Support Vector Machines (SVMs) are used for classification tasks, enhancing diagnostic accuracy by finding optimal boundaries between different classes. Random Forests, employed for both classification and regression, improve reliability by aggregating results from multiple decision trees. Neural Networks, including CNNs and RNNs, analyze complex image and sequence data, detecting subtle changes crucial for accurate diagnoses. Principal Component Analysis (PCA) and Linear Discriminant Analysis (LDA) simplify complex sensor data while preserving essential diagnostic information, speeding up processing and enhancing clarity.

The impact of ML on diagnostic outcomes is profound. Increased accuracy, achieved through effective data analysis and interpretation, leads to fewer false positives and negatives. ML enables personalized diagnostics by tailoring criteria to individual patient profiles and predictive analytics by forecasting trends for proactive healthcare interventions. By providing detailed insights, ML algorithms enhance decision support for healthcare professionals, facilitating informed diagnostic and treatment choices.

Machine learning (ML) algorithms find diverse applications across various sectors. In medical diagnostics, they aid in detecting diseases such as cancer, diabetes, and cardiovascular conditions by analyzing biosensor data and medical images. In industrial settings, ML enhances the performance of sensors used for monitoring equipment health and predicting maintenance needs, improving operational efficiency and reducing downtime.

The detection system in question distinguishes itself from existing technologies through several novel features and improvements. The use of laser-induced graphene (LIG) electrodes, offering superior electrochemical properties, enhances sensitivity and stability. The simplified one-step fabrication process streamlines production, reducing complexity and cost. Portability and user-friendliness are achieved through smartphone integration, enabling real-time testing and result delivery. The incorporation of advanced ML algorithms, such as SVM, Random Forests, and Neural Networks, improves data analysis accuracy. Additionally, the system offers affordable electrode production due to the simplified fabrication process and readily available materials.

The emphasis on portability and miniaturization in detection systems significantly expands point-of-care diagnostics. Increased accessibility and convenience are achieved through on-site testing, eliminating the need for central laboratories, and deployment in remote areas with limited access to traditional facilities. Rapid results and timely interventions are facilitated by immediate feedback, crucial for conditions requiring prompt treatment, and reduced waiting times, improving patient experience. Enhanced patient monitoring and management are enabled through regular monitoring of chronic conditions and personalized care plans. Cost-effectiveness is achieved by lowering infrastructure and logistics costs, potentially reducing the overall healthcare burden. Improved workflow and efficiency result from user-friendly device designs, enabling healthcare providers and patients to operate them with minimal training.

Yes, there are specific clinical settings and scenarios where this system is expected to have a particularly significant impact. For instance, in emergency departments, the system can facilitate rapid triage and diagnostic decisions, potentially reducing wait times and improving patient outcomes. In intensive care units (ICUs), the system's ability to monitor and analyze vast amounts of patient data in real-time can help detect early signs of deterioration, allowing for timely interventions.

More importantly, in outpatient clinics, the system can streamline administrative processes, such as patient scheduling and follow-up, enhancing overall efficiency and patient satisfaction. It can also support telemedicine by providing remote monitoring and diagnostic capabilities, which is particularly beneficial for patients in rural or underserved areas.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above about specific embodiments. However, the benefits, advantages, solutions to problems, and any component(s) that may cause any benefit, advantage, or solution to occur or become more pronounced are not to be construed as a critical, required, or essential feature or component of any or all the claims.

1. A miniaturized handheld electrochemical biosensor system for detecting creatinine from serum samples, the system comprises:
a laser-induced graphene (LIG) device to collect and retain serum samples, said LIG device comprises:
a flexible PI Sheet substrate;
at least three electrodes containing a reference electrode, a counter electrode, and a working electrode fabricated using a one-step fabrication process involving CO₂ Laser ablation on the polyimide sheet with optimized speed and power parameters;
a portable potentiostat having a conducting tract to electrically connect the LIG device to the portable potentiostat to detect creatinine in serum samples within a range of 60 μM/L to 110 μM/L for males and 45 to 90 μM/L for females in serum samples using an indirect electrochemical method; and
a graphical user interface coupled to a user computing device connected to the portable potentiostat to generate and display a graph upon receiving detected creatinine values from the portable potentiostat.

2. The system of claim 1, wherein the reference electrode is modified with silver-silver chloride ink (Ag/AgCl), the counter electrode and working electrode are modified with gold nanoparticles (AuNPs), and a picrate solution is placed after the modification of the working electrodes.

3. The system of claim 1, wherein the laser-induced graphene electrodes are obtained using a one-step fabrication process, wherein each graphene electrode comprises:
a substrate holder configured to hold a substrate;
a laser source configured to emit a laser beam;
a beam controller configured to control the laser beam and direct it onto the substrate; and
a processing unit configured to control the laser source and beam controller to perform laser scribing on the substrate for converting a portion of the substrate into graphene.

4. The system of claim 1, wherein the substrate comprises a polymer material, wherein the polymer material comprises polyimide.

5. The system of claim 3, further comprises:
a gas delivery unit configured to provide an inert atmosphere around the laser scribing region; and
a temperature control unit configured to maintain a desired temperature during the laser scribing process.

6. The system of claim 1, wherein a machine learning technique is incorporated to increase sensing and performance of the electrode with the various techniques selected from a group of regression-based accuracy matrices such as decision tree (DT), Linear Regression (LR), random forest (RF), K-Nearest Neighbor (kNN) and AdaBoost model.

7. The system of claim 1, wherein the LIG device contains enzymeless indirect sensing chemicals containing Picric acid and Sodium Hydroxide NaOH Solution., wherein the enzyme-less detection method works in a picrate anion consumed upon reaction with creatinine.

8. A method for detecting creatinine from serum samples using electrochemical biosensor system as claimed in claim 1, the method comprises:
providing a sample containing creatinine;
applying 10 μL sample droplet onto a surface of LIG electrodes of a laser-induced graphene (LIG) device;
establishing an electrical connection between the LIG electrodes and a portable detection system;
detecting creatinine in serum samples received from the LIG device through a conducting tract within a range of 60 μM/L to 110 μM/L for males and 45 to 90 μM/L for females in serum samples using an indirect electrochemical method using a portable potentiostat; and
generating a graph using a graphical user interface upon receiving detected creatinine values from the portable potentiostat thereby displaying on a user computing device.

9. The method of claim 8, further comprises fabricating miniaturized electrodes for creatinine detection, comprising the steps of:
providing a substrate, such as a flexible polyimide sheet;
directing a laser beam onto the substrate to perform laser scribing;
controlling the laser scribing process to convert a portion of the substrate into graphene, wherein the laser scribing process is performed in an inert atmosphere; and
patterning the graphene on the substrate by controlling the movement of the laser beam.

10. The method of claim 8, wherein the laser scribing process comprises:
controlling the laser power to achieve a desired level of conversion of the substrate to graphene;
controlling the scan speed of the laser beam to achieve a desired quality of the graphene;
controlling the ambient temperature during the laser scribing process;
optimizing the laser scribing process parameters to control the number of graphene layers formed; and
optimizing the laser scribing process parameters to minimize defects and ensure uniformity in the graphene layer.
